(19) United States
(12) Patent Application Publication
(10) Pub. No.: US 2023/0338270 A1
(43) Pub. Date: Oct. 26, 2023

(54) COSMETIC COMPOSITION FOR SKIN IMPROVEMENT COMPRISING, AS ACTIVE INGREDIENTS, POLYSACCHARIDES, YEAST EXTRACT, AND STRAIN FERMENTATION PRODUCT WITH CHARACTERISTICS OF PROBIOTICS

(71) Applicant: LG HOUSEHOLD & HEALTH CARE LTD., Seoul (KR)

(72) Inventors: Ji Yun KIM, Seoul (KR); Sa Do LEE, Seoul (KR); Sung Hwan HWANG, Seoul (KR)

(73) Assignee: LG HOUSEHOLD & HEALTH CARE LTD., Seoul (KR)

(21) Appl. No.: 17/799,450

(22) PCT Filed: Feb. 9, 2021
(86) PCT No.: PCT/KR2021/001717

§ 371 Date: Aug. 12, 2022

(30) Foreign Application Priority Data
Feb. 14, 2020 (KR) 10-2020-0018621
Feb. 3, 2021 (KR) 10-2021-0015590

(51) Int. Cl. A61K 8/9728; A61Q 7/00; A61Q 5/02; A61Q 19/08; A61K 8/73; A61K 8/99

(52) U.S. Cl. CPC A61K 8/732; A61K 8/9728; A61K 8/99; A61Q 5/02; A61Q 7/00; A61Q 19/08; A61K 2800/5922; A61K 2800/70; A61K 2800/85

(57) ABSTRACT

The present invention relates to a cosmetic composition, which is safe for the human body and has a skin condition improving effect by containing, as active ingredients, polysaccharides, a yeast extract, and a strain fermentation product with characteristics of probiotics. The cosmetic composition can be provided for improving microbial flora inhabiting the skin, soothing the skin, relieving skin wrinkles, improving skin elasticity, soothing the scalp, relieving the oiliness of the scalp, preventing hair loss, or improving hair growth.

### TECHNICAL FIELD

The present disclosure relates to an external application composition for skin improvement, including a polysaccharide, a yeast extract, and a strain fermentation product with probiotic characteristics as active ingredients.

### BACKGROUND ART

With a gradually increasing interest in health as well as beauty owing to improvement in the living standards, it is becoming common that people try to have beautiful skin, and various types of cosmetic compositions for skin care are being provided.

From this beauty point of view, hair takes a big part. Hair is a thin, keratinized structure produced on the surface of skin. It serves as a cushion against external shock, protects the human body from external stimuli such as direct sunlight, coldness, friction, and danger, and discharges, from the body, heavy metals such as arsenic, mercury, and zinc that are harmful to the body. Recently, an aesthetic aspect of hair in terms of decoration is becoming important as well.

Various factors, including changes in diet or increase in internal and external stress, are known to cause aggravation of hair condition including hair loss, but generally, since the scalp and hair are mutually dependent, the condition of scalp is known to be the most crucial factor affecting hair (Int. J. Trichology., 2018 Nov-Dec; 10(6): 262-270).

An external application composition for skin in addition to scalp and hair care has been provided in various forms based on raw materials, forms, and functions, most of which are added with artificial compounds to kill harmful bacteria on the skin or control sebum. Although such the artificial compound has excellent functional advantages, it sometimes irritates the skin and would rather cause damage to the skin in the long term.

Therefore, by overcoming the disadvantages of existing external application compositions for being applied to the skin, it is required to develop a composition that is safe for the human body and may effectively improve skin condition.

### DISCLOSURE OF THE INVENTION

### Technical Goals

The present disclosure has been made in an effort to solve the issues of the prior art as described above. Embodiments provide a cosmetic composition for skin improvement, capable of exhibiting effects of increasing beneficial bacteria or reducing harmful bacteria in the skin by including a plant-derived polysaccharide, a yeast-derived extract, and a strain fermentation product with probiotic characteristics as active ingredients.

### Technical Solutions

The present disclosure provides a cosmetic composition for skin improvement, including a polysaccharide, a yeast extract as a microorganism-derived extract, and a strain fermentation product with probiotic characteristics as a microorganism-derived fermented product as active ingredients.

The term “probiotics” as used herein refers to microorganisms that exist in the human body to have beneficial effects on human health and the actions thereof, and also includes all the metabolites such as dead cells, extracts, and fermented products of the microorganisms. The term “prebiotics” refers to food used for proliferation, growth promotion, and activity enhancement of the microorganisms.

The term “microflora” as used herein refers to a cell colony of microorganisms that are normally present in a specific part of the human body and may affect human health.

In embodiments of the present disclosure, the strain fermentation product with probiotic characteristics by itself did not show a significant effect on improvement in skin condition by increasing or reducing beneficial bacteria. However, it was found that a composition, further including polysaccharides that are probiotics and the yeast-derived extract as different type of probiotics in addition to the strain fermentation product, may exhibit probiotic characteristics capable of promoting growth and proliferation of beneficial bacteria, thereby providing the composition as a composition for improving scalp or skin condition.

In an embodiment of the present disclosure, the polysaccharide may be one or more selected from the group consisting of inulin, beta-glucan, and maltodextrin. The polysaccharides as described above contribute to the enhancement of immunity by improving the activity of immune cells in the human body and have the effect of reducing oxidative stress in cells.

Inulin is a polysaccharide that plays a role as storage for energy in many plants, such as chicory, wheat, onion, banana, garlic, and asparagus, and may generally be obtained from roots or rhizomes of the plants. When applied to the skin, inulin may protect microorganisms with probiotic characteristics existing in the skin against antibacterial substances. Inulin (C₆ₙH₁₀ₙ+₂O₅ₙ+₁) represented by the following Chemical Formula 1 includes a glucosyl moiety at a terminal end of a chain and a repeating fructosyl moiety therebetween.

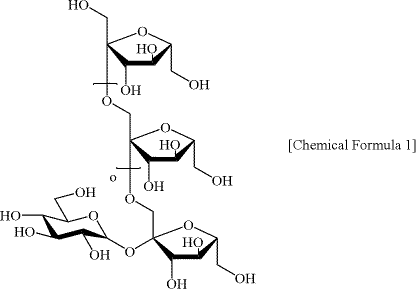

The n may be 2 to 60, but is not limited thereto. In an embodiment of present disclosure, inulin may be obtained from chicory roots, but may also be used by purchasing from domestic and foreign markets.

Beta-glucan is a polysaccharide formed in the cell walls of grains, bacteria, and fungi. When applied to the skin, beta-glucan has the effect of increasing antioxidant activities and anti-wrinkle activities in skin cells and improving UV protection, wound healing, and moisturizing functions. Beta-glucan represented by the following Chemical Formula 2 includes a repeating glucosyl moiety that forms a glycosidic bond at beta 1, 3 positions.

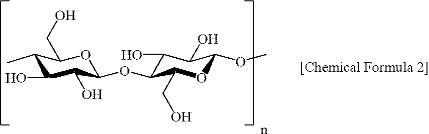

The n may be 2 to 100, but is not limited thereto. In an embodiment of the present disclosure, beta-glucan may be obtained by extraction from the fruiting body of mushroom, but may also be used by purchasing from domestic and foreign markets.

Maltodextrin is a polysaccharide formed by partial hydrolysis of vegetable starch. When applied to the skin, maltodextrin increases antioxidant activities in skin cells. Maltodextrin represented by the following Chemical Formula 3 includes a repeating glucosyl moiety that forms a glycosidic bond at alpha 1, 4 positions.

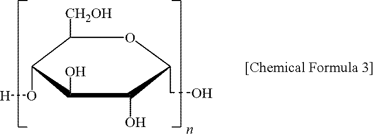

The n may be 2 to 20, but is not limited thereto. In the embodiment of the present disclosure, maltodextrin may be obtained by hydrolysis of corn or wheat starch, but may also be used by purchasing from domestic and foreign markets.

In an embodiment of the present disclosure, the yeast extract may refer to an extract obtained by extracting yeast with a solvent, a fraction obtainable by hydrolyzing yeast cells, a culture medium of yeast in which yeast and culture are present together, an extract extracted from the above-mentioned fraction or culture medium, a filtrate obtained by filtering yeast from the above-mentioned fraction or the culture medium, or a diluted solution or a dried product of the above-mentioned fraction or extract. For example, the yeast extract may be a fraction that is separated by a solvent after extracting yeast with a pharmaceutically acceptable organic solvent or hydrolyzing yeast cells using an enzyme.

In an embodiment of the present disclosure, the yeast extract may be one or more selected from the group consisting of a beer-derived yeast extract and a truffle-derived yeast extract.

The beer-derived yeast extract may be an extract obtained by culturing yeast in the genus Saccharomyces belonging to Ascomycetes or purchasing and culturing yeast of the same species, followed by extraction of the sterilized culture. Preferably, the yeast may be Saccharomyces cerevisiae. Brewer’s yeast, also called baker’s yeast, is used in the manufacture of alcoholic beverages including beer and bread. The beer-derived yeast extract may be obtained by drying yeast which is isolated after filtration of beer which is fermented by wort in the beer manufacturing process using the brewer’s yeast. The beer-derived yeast extract is non-fermentative and contains large amounts of carbohydrates, proteins, nucleic acids, and minerals such as vitamin B and phosphorus. Vitamin B group has the effect of improving skin health and hair condition. In an embodiment of the present disclosure, the beer-derived yeast extract may be obtained by isolating and culturing yeast used in the beer manufacturing process and sterilizing the same thereafter, but may also be used by purchasing from domestic and foreign markets.

The truffle-derived yeast extract may be an extract obtained by isolating and culturing yeast among microorganisms present in truffle or purchasing and culturing yeast of the same species found in truffle, followed by extraction of the sterilized culture. The truffle-derived yeast extract contains a large amount of beta-glucan and a small amount of vitamin B group. In an embodiment of the present disclosure, the truffle-derived yeast extract may be obtained by the method exemplified above, but may also be used by purchasing from domestic and foreign markets.

In an embodiment of the present disclosure, a strain with probiotic characteristics may be one or more anaerobic strains selected from the group consisting of Lactobacillus sp. strain and Bifidobacterium sp. strain. Therefore, a fermented product of the strain with probiotic characteristics may include fermentation metabolites of the strain, but is not limited thereto.

In an embodiment of the present disclosure, the fermented product may be fermented materials obtainable from strains as well as culture media in which the strain and culture are present together, a fermented product obtained by filtering the strain from the culture medium, a fermented product obtained by sterilizing the strain from the culture medium and filtering the same thereafter, an extract extracted from the above-mentioned fermented product or a culture medium including the same, a diluted solution or a dried product of the above-mentioned fermented product or extract, and a lysate obtained by collecting and crushing fungus bodies of the strain. Preferably, the fermented product may refer to a ferment lysate and a ferment filtrate.

For example, the fermented product may include ferment lysates and ferment filtrates. The ferment lysate refers to a product obtained by carrying out fermentation by anaerobic strains and then killing the bacteria using conditions such as heat, pH, enzyme, and pressure. The ferment lysate includes not only fermentation metabolites, but also various useful components in the strain. The ferment filtrate refers to a supernatant obtained by carrying out fermentation by anaerobic strains and then removing microorganisms including the strain by separation methods such as filtration.

Lactobacillus sp. strain is gram-positive bacteria that are distributed throughout the natural environment, such as the inside of the human body or plants and soil. The Lactobacillus sp. strain may produce lactic acid through fermentative metabolism for hexoses. The Lactobacillus sp. strain may suppress growth of harmful bacteria including specific pathogens by producing lactic acid or hydrogen peroxide as a product of metabolism in the human body.

In an embodiment of the present disclosure, the Lactobacillus sp. strain may include a homozygous lactic acid fermentation strain capable of fermenting a single hexose molecule into two lactic acid molecules as well as a heterozygous lactic acid fermentation strain capable of additionally producing ethanol, acetic acid, and carbon dioxide in addition to lactic acid molecules from hexose molecules. For example, the Lactobacillus sp. strain may include Lactobacillus delbrueckii (L. delbrueckii), Lactobacillus helveticus (L. helveticus), Lactobacillus salivarius (L. salivarius), Lactobacillus casei (L. casei), Lactobacillus plantarum (L. plantarum), Lactobacillus sakei (L. sakei), Lactobacillus brevis (L. brevis), Lactobacillus buchneri (L. buchneri), Lactobacillus fermentum (L. fermentum), Lactobacillus reuteri (L. reuteri), Lactobacillus rhamnosus (L. rhamnosus), Lacticaseibacillus paracasei (L. paracasei), Lactobacillus johnsonii (L. Johnsonii), and Lactobacillus bulgaricus (L. bulgaricus), but are not limited thereto. Preferably, the Lactobacillus sp. strain may be at least one of Lactobacillus acidophilus, Lactobacillus casei, Lactobacillus plantarum, Lactobacillus fermentum, Lactobacillus paracasei, and Lactobacillus bulgaricus. More preferably, the Lactobacillus sp. strain may be at least one of Lactobacillus acidophilus, Lactobacillus plantarum, Lactobacillus fermentum, and Lactobacillus paracasei. Most preferably, the Lactobacillus sp. strain may be at least one of Lactobacillus acidophilus and Lactobacillus plantarum.

Therefore, in an embodiment of the present disclosure, a fermented product of Lactobacillus sp. strain may include the fermentation metabolite of the strain for hexoses, and preferably be a ferment lysate obtained by killing the strain.

Bifidobacterium sp. strain is gram-positive bacteria that are distributed throughout the natural environment, such as skin of human body, food, and soil. The Bifidobacterium sp. strain is a heterogeneous lactic acid fermentation strain that produces lactic acid, acetic acid, and formic acid through a unique hexose fermentation metabolism using fructose-6-phosphate phosphoketolases.

In an embodiment of the present disclosure, the Bifidobacterium sp. strain may include, for example, Bifidobacterium animalis (B. animalis), Bifidobacterium bifidum (B. bifidum), Bifidobacterium breve (B. breve), Bifidobacterium dentium (B. dentium), and Bifidobacterium longum (B. longum), but are not limited thereto. Preferably, the Bifidobacterium sp. strain may be at least one of Bifidobacterium animalis, Bifidobacterium bifidum, and Bifidobacterium longum.

In an embodiment of the present disclosure, a fermented product of Bifidobacterium sp. strain may include fermentation metabolites of the strain for hexoses, and preferably be a ferment lysate obtained by killing the strain.

The cosmetic composition for skin improvement in accordance with an embodiment of the present disclosure may include inulin, beta-glucan, maltodextrin, the beer-derived yeast extract, the truffle-derived yeast extract, a Lactobacillus ferment lysate, or a Bifidobacterium ferment lysate as an active ingredient.

Preferably, the cosmetic composition may include inulin, beta-glucan, maltodextrin, the beer-derived yeast extract, the truffle-derived yeast extract, the Lactobacillus ferment lysate, and the Bifidobacterium ferment lysate in the same ratio, respectively. For example, the cosmetic composition may include inulin, beta-glucan, maltodextrin, the beer-derived yeast extract, the truffle-derived yeast extract, the Lactobacillus ferment lysate, and the Bifidobacterium ferment lysate in a weight ratio of a:b:c:d:e:f:g in order. Here, a to g may be 1 to 10 respectively, preferably, a to g may be 1 to 5 respectively, and most preferably, a to g may all be 1.

The cosmetic composition for skin improvement in accordance with an embodiment of the present disclosure may include one or more active ingredients selected from the group consisting of inulin, beta-glucan, maltodextrin, the beer-derived yeast extract, the truffle-derived yeast extract, the Lactobacillus ferment lysate, and the Bifidobacterium ferment lysate in an amount of 0.00001 to 10 wt% with respect to the total of 100 wt%. Preferably, the content may be 0.0001 to 1 wt%, more preferably 0.0005 to 0.5 wt%, and most preferably 0.001 to 0.1 wt%.

The term “skin improvement” as used herein may refer to not only improvement in skin condition observed with naked eyes, but also factors that directly and indirectly affect skin health.

Specifically, the cosmetic composition for skin improvement in accordance with an embodiment of the present disclosure may be for improving microbial flora inhabiting the skin. The improvement of microbial flora may include increasing beneficial bacteria by promoting the growth of beneficial bacteria present in the skin or reducing harmful bacteria by suppressing the growth of harmful bacteria.

Specifically, the cosmetic composition for skin improvement in accordance with an embodiment of the present disclosure may be any one of skin soothing, skin wrinkle amelioration, and skin elasticity improvement. Skin soothing may include prevention or normalization of disorders on the skin, in addition to prevention or suppression of inflammation in the skin. Skin wrinkle amelioration or elasticity improvement may include prevention or suppression of damage or volume reduction in the dermal layer caused by natural factors such as aging as well as external environmental factors such as UV rays and oxidative stress.

Specifically, the cosmetic composition for skin improvement in accordance with an embodiment of the present disclosure may be for being applied to the scalp. For example, the cosmetic composition for scalp improvement may be any one of scalp soothing, scalp oil improvement, hair loss prevention, and hair growth promotion. Skin soothing may refer to prevention or suppression of inflammation in the scalp. Scalp oil improvement may refer to reduction in an amount of oil secretion when excess oil is secreted from the skin. Hair loss prevention or hair growth promotion may include increasing the activity of dermal papilla cells in the scalp, inhibiting inflammatory factors for cells constituting the skin, or increasing the collagen biosynthesis rate in cells constituting the skin.

Preferably, “skin improvement” may refer to an increase in beneficial bacteria present in the skin and a decrease in harmful bacteria. The beneficial bacteria and harmful bacteria are classified based on the effect of strains regarding the skin health of the human body. For example, it may include a Staphylococcus sp. strain, but is not limited thereto.

For example, of the Staphylococcus sp. strains, bacteria such as Staphylococcus epidermidis do not act as pathogens for normal skin, but produce antibacterial peptides against other pathogens or inhibit the progression of some cancers, thereby being classified as beneficial bacteria.

For example, of the Staphylococcus sp. strains, bacteria such as Staphylococcus aureus exist on the skin surface or pores and cause various infections in the skin as well as diseases even in normal skin, thereby being classified as harmful bacteria. If the skin is scalp, harmful bacteria may include Aspergillus niger (A. niger), which may cause inflammation in the scalp by producing mycotoxins.

As will be described later, the composition in accordance with an embodiment of the present disclosure may provide an improvement effect on skin by bringing enhancement in the flora in the human body, such as an increase in beneficial bacteria.

Human dermal papilla cells are mesenchymal cells present in hair follicles in the scalp and play a decisive role in controlling the events regarding hair generation and hair growth cycles. Excessive oil in the scalp may cause dermatitis, and inflammatory factors may induce an inflammatory response against cells present in the scalp. Collagen refers to a protein constituting connective tissues present in the extracellular matrix, and a decrease in the collagen biosynthesis rate in the scalp may cause weakening of dermis of the scalp and shrinkage of hair follicles thereby.

As will be described later, the composition in accordance with an embodiment of the present disclosure provides an improvement effect on the skin by increasing the activity of specific cells, reducing oil in the skin, inhibiting the production of inflammatory factors in the skin cells, and increasing the collagen biosynthesis rate.

The term “cosmetic composition” as used herein refers to a composition for being administered by direct application or spraying to the skin. The term “administration” as used herein refers to introduction of the composition in accordance with an embodiment of the present disclosure to a target tissue using a conventional method known in the art.

The cosmetic composition for skin improvement in accordance with an embodiment of the present disclosure may have a formulation of toner, facial lotion, body lotion, nourishing cream, moisture cream, eye cream, essence, cosmetic ointment, spray, gel, pack, sunscreen, makeup base, foundation, powder, cleansing cream, cleansing lotion, cleansing oil, cleansing foam, soap, or body wash.

In the cosmetic composition for skin improvement in accordance with an embodiment of the present disclosure, when the skin is scalp or hair, it may have a formulation of hair tonic, hair conditioner, hair essence, hair lotion, hair nutrition lotion, hair shampoo, hair rinse, hair treatment, hair cream, hair nutrition cream, hair moisture cream, hair massage cream, hair wax, hair aerosol, hair pack, hair nutrition pack, hair soap, hair cleansing foam, hair oil, hair dryer agent, hair preservative, hair dye, hair wave agent, hair bleach, hair gel, hair glaze, hair dresser, hair lacquer, hair moisturizer, hair mousse, or hair spray.

When the composition of an embodiment of the present disclosure is formulated in a liquid, the composition of an embodiment of the present disclosure may include a carrier for the active ingredient. The carrier may include, for example, water, ethanol, castor oil, glycerin, isopropanol, ethyl carbonate, ethyl acetate, benzyl alcohol, benzyl benzoate, propylene glycol, 1,3-butyl glycol oil, glycerol aliphatic ester, or fatty acid ester such as polyethylene glycol or sorbate, but is not limited thereto. These may be used alone or in combination of two or more.

When the composition of an embodiment of the present disclosure is formulated as a paste, cream, or gel, the composition of an embodiment of the present disclosure may include a carrier for the active ingredient. The carrier may include, for example, animal oil, vegetable oil, wax, paraffin, starch, cellulose derivatives such as tragacanth and hydroxyethyl, polyethylene glycol, silicon, bentonite, silica, talc, zinc oxide, cetostearyl alcohol, or chloride stearyl trimethylammonium, but is not limited thereto. These may be used alone or in combination of two or more.

When the composition of an embodiment of the present disclosure is formulated as powder or spray, the composition of an embodiment of the present disclosure may include a carrier for the active ingredient. The carrier may include, for example, lactose, talc, silica, aluminum hydroxide, calcium silicate, or polyamide powder, but is not limited thereto. These may be used alone or in combination of two or more. When the composition of an embodiment of the present disclosure is formulated as a spray, a propellant such as chlorofluorohydrocarbon, propane, butane, or dimethyl ether may be further included.

When the composition of an embodiment of the present disclosure is formulated as soap, the composition of an embodiment of the present disclosure may include a carrier for the active ingredient. The carrier may include, for example, an alkali metal salt of fatty acid, a fatty acid hemiester salt, a fatty acid protein hydrolysate, isethionate, a lanolin derivative, aliphatic alcohol, vegetable oil, glycerol, and sugar, but is limited thereto. These may be used alone or in combination of two or more.

The composition of an embodiment of the present disclosure may further include additives commonly used for cosmetic compositions. The additive may include, for example, purified water, a surfactant, a moisturizer, lower alcohol, a chelating agent, a disinfectant, an antioxidant, a preservative, a color, or a fragrance, but is not limited thereto. These may be used alone or in combination of two or more.

The present disclosure provides a use of one or more of a polysaccharide, a yeast extract, and a strain fermentation product with probiotic characteristics. Specifically, the present disclosure provides a use of one or more of a polysaccharide, a yeast extract, and a strain fermentation product with probiotic characteristics in a cosmetic composition for skin improvement. More specifically, the present disclosure provides a use of one or more of a polysaccharide, a yeast extract, and a strain fermentation product with probiotic characteristics in a method for preparing a cosmetic composition for skin improvement.

In addition, the present disclosure provides a use of one or more of a polysaccharide, a yeast extract, and a strain fermentation product with probiotic characteristics in a skin improvement method, a method for improving the microbial flora inhabiting the skin, a skin soothing method, a skin wrinkle amelioration method, a skin elasticity improvement method, a scalp soothing method, a scalp oil improvement method, a hair loss prevention method, or a hair growth promotion method.

In addition, the present disclosure provides a method for improving skin, a method for improving the microbial flora inhabiting the skin, a method for soothing skin, a method for ameliorating skin wrinkles, a method for improving skin elasticity, a method for calming the scalp, a method for reducing scalp oil, a method for preventing hair loss, or a method for promoting hair growth, by treating a subject with a cosmetic product including one or more of a polysaccharide, a yeast extract, and a strain fermentation product with probiotic characteristics.

All components included in embodiments of the present disclosure do not exceed the regulations set by each country. Preferably, the cosmetic composition of an embodiment of the present disclosure may contain the individual components mentioned above within a range not exceeding the maximum dosage stipulated in the “Safety and Technical Standard for Cosmetics” set by the Chinese government.

### Advantageous Effects

A cosmetic composition for skin improvement in accordance with an embodiment of the present disclosure includes all polysaccharides as prebiotics, a yeast-derived extract as probiotics, and a fermented product, thereby attaining outstanding effects for proliferation of beneficial bacteria and suppression of harmful bacteria in the skin, compared to a case when prebiotics or probiotics are solely applied. Such the cosmetic composition is harmless to the human body and has the effect of improving the skin condition through improvement in flora in the skin.

Specifically, the cosmetic composition for skin improvement in accordance with an embodiment of the present disclosure has the effect of improving scalp health and hair condition by increasing the activity of dermal papilla cells.

In addition, the cosmetic composition for skin improvement in accordance with an embodiment of the present disclosure has the effect of improving skin condition by suppressing the generation of inflammatory factors for skin cells.

In addition, the cosmetic composition for skin improvement in accordance with an embodiment of the present disclosure has the effect of improving condition and health of skin by increasing the collagen biosynthesis rate in skin cells.

In addition, the cosmetic composition for skin improvement in accordance with an embodiment of the present disclosure has the effect of improving the oil-water balance on the skin surface by removing foreign substances and oil without adverse effects on the skin.

### BRIEF DESCRIPTION OF DRAWINGS

FIGS. 1 shows photographs of scalp (A) before application and (B) after a single application of a hair shampoo in accordance with an embodiment of the present disclosure.

FIGS. 2 shows photographs of scalp (A) before application and (B) after a single application of a hair shampoo in accordance with an embodiment of the present disclosure.

FIGS. 3 shows photographs of scalp (A) before application and (B) after a single application of a hair shampoo in accordance with an embodiment of the present disclosure.

### BEST MODE FOR CARRYING OUT THE INVENTION

The objects, specific advantages, and novel features of an embodiment of the present disclosure will become more apparent from the following detailed description and preferred examples. However, these examples are for illustrative purposes only, and the scope of the present disclosure is not limited to these examples. In addition, in describing the present disclosure, if it is determined that a detailed description of a related known technology may unnecessarily obscure the gist of the present disclosure, the detailed description thereof will be omitted.

Manufacturing example. Complex including polysaccharides, yeast extract, and strain fermentation product with probiotic characteristics

Used in the following examples were commercially available products including inulin (Beneo), beta-glucan (Quegen Biotech Co.,Ltd.), maltodextrin (Samyang), and a beer-derived yeast extract (Activon). Used as a truffle-derived yeast extract was an extract extracted from a product obtained by isolating yeast from truffle and performing culture at 37° C. for 48 hours, followed by sterilization. Used as a Lactobacillus ferment lysate and a Bifidobacterium ferment lysate were an extract extracted from a product obtained by culturing Lactobacillus and Bifidobacterium (Chr. Hansen) at 37° C. for 48 hours and then sterilizing the same.

(Preparation Example 1) A complex was prepared by mixing each 10 g of inulin, the beer-derived yeast extract, and the Lactobacillus ferment lysate. (Preparation Example 2) A complex was prepared by mixing each 10 g of beta-glucan, the beer-derived yeast extract, and the Lactobacillus ferment lysate. (Preparation Example 3) A complex was prepared by mixing each 10 g of maltodextrin, the beer-derived yeast extract, and the Lactobacillus ferment lysate. (Preparation Example 4) A complex was prepared by mixing each 10 g of inulin, the truffle-derived yeast extract, and the Bifidobacterium ferment lysate. (Preparation Example 5) A complex was prepared by mixing each 10 g of beta-glucan, the truffle-derived yeast extract, and the Bifidobacterium ferment lysate. (Preparation Example 6) A complex was prepared by mixing each 10 g of maltodextrin, the truffle-derived yeast extract, and the Bifidobacterium ferment lysate. (Preparation Example 7) A complex was prepared by mixing each 10 g of inulin, beta-glucan, maltodextrin, the beer-derived yeast extract, the truffle-derived yeast extract, the Lactobacillus ferment lysate, and the Bifidobacterium ferment lysate.

Example. Cosmetic composition including polysaccharide, yeast extract, and strain fermentation product with probiotic characteristics as active ingredients

### Example 1-1. Hair Shampoo Composition

A hair shampoo composition having a composition as shown in Table 1 below was prepared.

TABLE 1

| Ingredient name | Content (wt%) | Ingredient name | Content (wt%) |
|---|---|---|---|
| Disodium laureth sulfosuccinate | 5.000 | Inulin | 0.001 |
| Lauryl hydroxysultaine | 5.000 | Beta-glucan | 0.001 |
| Disodium lauryl sulfosuccinate | 1.000 | Maltodextrin | 0.001 |
| Glycerin | 1.000 | Beer-derived yeast extract | 0.001 |
| Caprylyl glycol | 1.000 | Truffle-derived yeast extract | 0.001 |
| Tetrasodium EDTA | 0.500 | Lactobacillus ferment lysate | 0.001 |
| Spices | Appropriate amount | Bifidobacterium ferment lysate | 0.001 |
| pH adjuster | Appropriate amount | Purified water | Remaining amount |
| Preservative | Appropriate amount | | |
| Total | | | 100 |

### Example 1-2. Hair Shampoo Composition

A hair shampoo composition having a composition as shown in Table 2 below was prepared.

TABLE 2

| Ingredient name | Content (wt%) | Ingredient name | Content (wt%) |
|---|---|---|---|
| Disodium laureth sulfosuccinate | 10.000 | Inulin | 0.001 |
| Cocamidopropyl | 5.000 | Beta-glucan | 0.001 |
| betaine | | | |
| Glycerin | 1.000 | Maltodextrin | 0.001 |
| Caprylyl glycol | 1.000 | Beer-derived yeast extract | 0.001 |
| Polyquaternium-7 | 0.500 | Truffle-derived yeast extract | 0.001 |
| Spices | Appropriate amount | Lactobacillus ferment lysate | 0.001 |
| pH adjuster | Appropriate amount | Bifidobacterium ferment lysate | 0.001 |
| Preservative | Appropriate amount | Purified water | Remaining amount |
| Total | | | 100 |

### Example 2. Hair Treatment Composition

A hair treatment composition having a composition as shown in Table 3 below was prepared.

TABLE 3

| Ingredient name | Content (wt%) | Ingredient name | Content (wt%) |
|---|---|---|---|
| Ethanol | 1.000 | Inulin | 0.001 |
| Cetearyl alcohol | 20.000 | Beta-glucan | 0.001 |
| Stearamidopropyl dimethylamine | 5.000 | Maltodextrin | 0.001 |
| Shea butter | 5.000 | Beer-derived yeast extract | 0.001 |
| Macadamia seed oil | 5.000 | Truffle-derived yeast extract | 0.001 |
| PEG-100 stearate | 5.000 | Lactobacillus ferment lysate | 0.001 |
| Polyquaternium-7 | 0.500 | Bifidobacterium ferment lysate | 0.001 |
| pH adjuster | Appropriate amount | Spices | Appropriate amount |
| Preservative | Appropriate amount | Purified water | Remaining amount |
| Total | | | 100 |

### Example 3. Hair Tonic

A hair tonic composition having a composition as shown in Table 4 below was prepared.

TABLE 4

| Ingredient name | Content (wt%) | Ingredient name | Content (wt%) |
|---|---|---|---|
| Ethanol | 30.000 | Octyldodeces-16 | 0.200 |
| Lightyellow | 0.005 | Inulin | 0.001 |
| Sophora root extract | | | |
| Niacinamide | 0.100 | Beta-glucan | 0.001 |
| Menthol | 0.100 | Maltodextrin | 0.001 |
| Guinea pepper extract | 0.050 | Beer-derived yeast extract | 0.001 |
| Benzyl nicotinate | 0.005 | Truffle-derived yeast extract | 0.001 |
| Asarum canadense root extract | 0.300 | Lactobacillus ferment lysate | 0.001 |
| Spices | Appropriate amount | Bifidobacterium ferment lysate | 0.001 |
| pH adjuster | Appropriate amount | Purified water | Remaining amount |
| Total | | | 100 |

### Example 4. Body Wash

A body wash composition having a composition as shown in Table 5 below was prepared.

TABLE 5

| Ingredient name | Content (wt%) | Ingredient name | Content (wt%) |
|---|---|---|---|
| Trisodium EDTA | 0.050 | Inulin | 0.001 |
| Disodium cocoyl glutamate | 4.000 | Beta-glucan | 0.001 |
| Sodium cocoyl alaninate | 3.000 | Maltodextrin | 0.001 |
| Lauramidopropyl betaine | 3.000 | Beer-derived yeast extract | 0.001 |
| PEG-40 hydrogenated castor oil | 1.000 | Truffle-derived yeast extract | 0.001 |
| Spices | Appropriate amount | Lactobacillus ferment lysate | 0.001 |
| pH adjuster | Appropriate amount | Bifidobacterium ferment lysate | 0.001 |
| Preservative | Appropriate amount | Purified water | Remaining amount |
| Total | | | 100 |

### Example 5. Body Lotion

A body lotion composition having a composition as shown in Table 6 below was prepared.

TABLE 6

| Ingredient name | Content (Wt%) | Ingredient name | Content (Wt%) |
|---|---|---|---|
| Glycerin | 8.000 | Inulin | 0.001 |
| Caprylic/Capric triglycerides | 2.000 | Beta-glucan | 0.001 |
| Cetyl ethylhexanoate | 2.000 | Maltodextrin | 0.001 |
| Glyceryl stearate | 2.000 | Beer-derived yeast extract | 0.001 |
| Cetearyl alcohol | 1.000 | Truffle-derived yeast extract | 0.001 |
| Acrylate/C10-30 alkyl acrylate crosspolymer | 0.100 | Lactobacillus ferment lysate | 0.001 |
| Disodium EDTA | 0.020 | Bifidobacterium ferment lysate | 0.001 |
| pH adjuster | Appropriate amount | Spices | Appropriate amount |
| Preservative | Appropriate amount | Purified water | Remaining amount |
| Total | | | 100 |

### Experimental Example 1. Identification of the Effect on the Growth of Beneficial Bacteria

In order to identify the effect of complexes of Preparation Examples on the growth of beneficial bacteria, Staphylococcus epidermidis (S. epidermidis) was selected as beneficial bacteria and Staphylococcus aureus (S. aureus) as harmful bacteria. Before use, the bacteria were stored at -80° C., a plurality of 100 mL tryptic soy broth (TSB) media was prepared, and 500 µl of each bacterium stock was inoculated for each medium. After rotary culture at a rate of 210 rpm at 37° C. for 17 hours, 4 mL of the culture medium was collected to be inoculated into 40 mL of TSB, followed by subculture at a rate of 210 rpm at 37° C. for 8 hours.

After 8 hours, the optical density (O.D.) value was adjusted to 0.65, complexes prepared according to Preparation Examples 1 to 7 were inoculated at a concentration of 1%, respectively, and initial optical density values were measured. Thereafter, the optical density was measured after rotary culture at a rate of 210 rpm at 37° C. for 16 hours. The difference between the optical density measurement value and the initial measurement value after 16 hours was standardized for each medium by dividing by the initial measurement value for evaluation.

Used as comparative examples were inulin, beta-glucan, maltodextrin, the beer-derived yeast extract, the truffle-derived yeast extract, the Lactobacillus ferment lysate, and the Bifidobacterium ferment lysate, each inoculated at a concentration of 1%. An untreated group that was treated with none was used as a control group.

The inoculation results of the Preparation Examples and Comparative Examples were compared to the untreated group, and the average degree of increase or decrease of beneficial bacteria and harmful bacteria was converted into percentages. The results are shown in Table 7 below.

TABLE 7

| Classification | Beneficial bacteria (S. epidermidis) | Harmful bacteria (S. aureus) |
|---|---|---|
| Lactobacillus ferment lysate | -9.90 | 21.42 |
| Bifidobacterium ferment lysate | -4.69 | 26.00 |
| Beer-derived yeast extract | 2.48 | 16.37 |
| Truffle-derived yeast extract | 3.39 | 14.95 |
| Inulin | 0.29 | -0.84 |
| Beta-glucan | -0.26 | -0.78 |
| Maltodextrin | 0.14 | 1.43 |
| Preparation Example 1 | 5.51 | -2.46 |
| Preparation Example 2 | 4.79 | -2.88 |
| Preparation Example 3 | 3.88 | -2.31 |
| Preparation Example 4 | 7.18 | -2.49 |
| Preparation Example 5 | 6.07 | -2.71 |
| Preparation Example 6 | 5.99 | -2.19 |
| Preparation Example 7 | 15.54 | -7.47 |

As shown in Table 7, although inulin, beta-glucan, and maltodextrin are polysaccharides with prebiotic characteristics, no significant effect was shown alone on the increase in beneficial bacteria and reduction in harmful bacteria. The ferment lysate with probiotic characteristics alone rather showed effects of decreasing beneficial bacteria while reducing harmful bacteria.

In contrast, the complexes according to Preparation Examples 1 to 7 of the present disclosure showed significantly outstanding effects in increasing beneficial bacteria and reducing harmful bacteria compared to Comparative Examples in which the polysaccharides, the yeast extract, or the ferment lysate were used alone. When the polysaccharides as prebiotics as well as the yeast extract and the ferment lysate as probiotics were used together, it was found that there was a synergistic effect in the increase in beneficial bacteria and the decrease in harmful bacteria. In particular, it was observed that Preparation Example 7 showed the most outstanding effect on the increase in beneficial bacteria and the increase in harmful bacteria compared to other Preparation Examples.

### Experimental Example 2. Identification of Activity of Dermal Papilla Cells

In order to identify the effect of the complexes of Preparation Examples on dermal papilla cells, human dermal papilla cells (Promocell, C-12071) in the 4-8 passage were used as a cell line, and follicle dermal papilla cell growth media (Promocell, C-26501) were used as an initial culture medium.

Cells were seeded at a level of 3,000 to 6,000 cells per well in a 96-well plate, and cultured in an incubator at 37° C. in the presence of 5% carbon dioxide for 24 hours. Thereafter, the culture medium was replaced with DMEM medium supplemented with 0.1% fetal bovine serum (FBS) and cultured in an incubator at 37° C. in the presence of 5% carbon dioxide for 24 hours. Thereafter, the culture medium was replaced with DMEM supplemented with 0.1% FBS treated with the complexes according to Preparation Examples 1 to 7 by each concentration, followed by culture in an incubator at 37° C. in the presence of 5% carbon dioxide for 48 hours. After treating 10 µl of CCK-8 solutions per well, culture was performed at 37° C. for 1 hour, and optical density was measured at 450 nm.

The difference between the optical density measurement value and the initial measurement value after 48 hours for each well was standardized by dividing the difference by the initial measurement value for evaluation, and the average value was shown in Table 8 below. 1 µM minoxidil was treated as a control group. For a significant difference, p-value was derived by assuming equal variance (significant difference p<0.05).

TABLE 8

| | Concentration | Average |
|---|---|---|
| Preparation Example 1 | 1 ppm | 5.8 |
| | 10 ppm | 7.7 |
| Preparation Example 2 | 1 ppm | 5.6 |
| | 10 ppm | 7.1 |
| Preparation Example 3 | 1 ppm | 5.2 |
| | 10 ppm | 8.1 |
| Preparation Example 4 | 1 ppm | 4.8 |
| | 10 ppm | 7.7 |
| Preparation Example 5 | 1 ppm | 6.1 |
| | 10 ppm | 10.2 |
| Preparation Example 6 | 1 ppm | 5.9 |
| | 10 ppm | 8.8 |
| Preparation Example 7 | 1 ppm | 16.9 |
| | 10 ppm | 21.5 |
| Minoxidil | 1 µM | 44.2 |

As shown in Table 8, it was found that the complexes according to Preparation Examples 1 to 7 of an embodiment of the present disclosure had a synergistic effect on dermal papilla cell activity. In particular, it was found that Preparation Example 7 showed the most outstanding effect on the increase in dermal papilla cell activity compared to other Preparation Examples.

### Experimental Example 3. Identification of Suppression of Inflammatory Factors

In order to identify the effect of the complexes of Preparation Examples on the production of NO as an inflammatory factor in cells, a mouse Raw 264.7 cell line was used, and a culture medium whose final pH was adjusted to 8.5 through addition of NaOH to Complete RPMI (RPMI:FBS:antibiotic = 10:1:0.1) was used as an initial culture medium.

When the Raw 264.7 cells appeared at the top of the culture medium after culture at 37° C. in the presence of 5% carbon dioxide for 24 hours, the cells were seeded in a 24-well plate in which a DMEM culture was prepared, followed by culture at 37° C. in the presence of 5% carbon dioxide for 24 hours. Thereafter, treatment was performed after diluting the complexes according to Preparation Examples 1 to 7 by the following concentrations while replacing the medium with a serum-free DMEM culture medium, followed by additional treatment of 1 µg/mL of LPS. After culture at 37° C. in the presence of 5% carbon dioxide for 24 hours, the supernatant was mixed with Griess reagent (Sigma-Aldrich) in a ratio of 1:1 to evaluate the ability to inhibit NO production with absorbance, and the average value was shown in Table 9 below.

NO production inhibition ability (%) = {1 - (NO production when compound is added / NO production in untreated group)} × 100

20 µg/mL of L-NMMA1 (N^{G}-Methyl-L-arginine acetate salt), an NO inhibitor, was used as a control group.

TABLE 9

| | Concentration | Average |
|---|---|---|
| Preparation Example 1 | 1% | 2.2 |
| | 7% | 10.8 |
| Preparation Example 2 | 1% | 3.1 |
| | 7% | 7.5 |
| Preparation Example 3 | 1% | 3.5 |
| | 7% | 8.1 |
| Preparation Example 4 | 1% | 2.8 |
| | 7% | 9.4 |
| Preparation Example 5 | 1% | 3.3 |
| | 7% | 8.7 |
| Preparation Example 6 | 1% | 3.3 |
| | 7% | 7.5 |
| Preparation Example 7 | 1% | 5.7 |
| | 7% | 23.3 |
| L-NMMA1 | 20 µg/mL | 52.1 |

As shown in Table 9, it was found that the complexes according to Preparation Examples 1 to 7 of an embodiment of the present disclosure had the ability to inhibit NO production as an inflammatory factor in cells. In particular, it was found that Preparation Example 7 showed the most excellent NO production inhibitory ability compared to other Preparation Examples.

### Experimental Example 4. Identification of Collagen Biosynthesis Rate

The effect of the complexes of Preparation Examples on the collagen biosynthesis rate in the human skin fibroblasts was identified. Skin fibroblasts were cultured in complete DMEM culture at 37° C. in the presence of 5% carbon dioxide for 72 hours. After seeding in a 24-well plate, culture was performed at 37° C. in the presence of 5% carbon dioxide for 24 hours. After treating the complexes according to Preparation Examples and culturing at 37° C. in the presence of 5% carbon dioxide for 48 hours, only the supernatant was separated, and the collagen synthesis effect was observed by absorbance using a procollagen type I C-peptide (PIP) EIA kit. The increase rate of collagen biosynthesis was evaluated as follows, and the average value was shown in Table 10 below:

Collagen biosynthesis increase rate (%) = {(Absorbance of complex treated group -Absorbance of untreated group) / Absorbance of untreated group} × 100

10 ng/mL of TGF-β, a cytokine, was used as a control group.

TABLE 10

| | Concentration | Average |
|---|---|---|
| Preparation Example 1 | 1% | 8.7 |
| | 7% | 10.8 |
| Preparation Example 2 | 1% | 8.6 |
| | 7% | 11.5 |
| Preparation Example 3 | 1% | 7.1 |
| | 7% | 10.8 |
| Preparation Example 4 | 1% | 9.5 |
| | 7% | 12.2 |
| Preparation Example 5 | 1% | 10.3 |
| | 7% | 15.4 |
| Preparation Example 6 | 1% | 8.5 |
| | 7% | 11.1 |
| Preparation Example 7 | 1% | 15.0 |
| | 7% | 35.5 |
| TGF-β | 10 ng/mL | 36.6 |

As shown in Table 10, it was found that the complexes according to Preparation Examples 1 to 7 of an embodiment of the present disclosure had an effect of increasing collagen biosynthesis in fibroblasts. In particular, it was found that Preparation Example 7 showed the most excellent effect on the increase in collagen biosynthesis in fibroblasts compared to other Preparation Examples.

### Experimental Example 5. Checking Whether Scalp Oil is Improved

The scalp oil improvement effect when the hair shampoo composition according to Example 1-1 was used was identified. As test subjects, 22 adult women aged 20 to 50 years were chosen, whose scalp was selected as the test site. The test subjects were not allowed to use other products such as shampoos, treatments, and hair essences and get hair dyed or permed during the experiment.

The experiment was performed after allowing the test subject to take a rest for 30 minutes in a constant temperature and humidity room at 22±2° C. and 50±5% humidity, and all experiments were performed in the constant temperature and humidity room. After wetting the hair and scalp of the test subject sufficiently with lukewarm water, the same amount of the hair shampoo composition according to Example 1-1 was evenly applied to the hair and scalp. After massage, rinsing was followed thoroughly with running water.

The evaluation for scalp oil before and after the use of the hair shampoo was performed using a Sebumeter (SKIN-O-MAT, Cosmomed GmbH) and a video microscope (Kong PC Camera, Bomtech). A probe cassette with an oil adsorption tape attached was placed on the top of the head of all test subjects, and the oil was sufficiently absorbed by brining the cassette in contact with the same pressure for 30 seconds. Then, the cassette was inserted into a main body of the Sebumeter to measure the amount of sebum. In addition, under the same lighting condition, the top of the head of all test subjects was observed at 300 times magnification using a video microscope.

Hereinafter, the analysis was conducted using SPSS 17.0 for Windows for statistical processing in this experiment. Table 11 below is a result showing the amount of sebum (µg/cm²) before use and after a single use of the composition of Example 1-1 (the maximum amount of sebum measurable with a Sebumeter is 350 µg/cm²).

TABLE 11

| | Before use | Immediately after a single use |
|---|---|---|
| Average | 200.41 | 32.05 |
| Standard Deviation | 37.03 | 14.87 |

Table 12 below shows the results of paired t-test analysis (*p<0.05, **p<0.01, ***p<0.001) for analyzing the sebum amount improvement rate (%) and significant change after a single use of the composition of Example 1-1: Improvement rate (%) = {(Measured value after 1 time use - Measured value before use) / Measured value before use} × 100

TABLE 12

| | Sebum improvement rate | p-value |
|---|---|---|
| Immediately after a single use | 84.01 | 0.001 *** |

In addition, scalp condition of the individual test subject and changes in the scalp condition before use and after a single use of the composition of Example 1-1 were surveyed, and the results were shown in Tables 13 and 14 below, respectively (N = Total frequency = Number of subjects = 22).

TABLE 13

| Question | | Frequency | Percentage (%) |
|---|---|---|---|
| The scalp is clean with no wastes or foreign substances. | Not at all | 2 | 9.1 |
| | No | 19 | 86.4 |
| | Indifferent | 1 | 4.5 |
| | Yes | 0 | 0.0 |
| | Excellent | 0 | 0.0 |
| The scalp is fresh without excessive sebum secretion. | Not at all | 2 | 9.1 |
| | No | 19 | 86.4 |
| | Indifferent | 1 | 4.5 |
| | Yes | 0 | 0.0 |
| | Excellent | 0 | 0.0 |
| Total | | 22 | 100.0 |

TABLE 14

| Question | | Frequency | Percentage (%) |
|---|---|---|---|
| Wastes and foreign substances on the scalp seem to be | Not at all | 0 | 0.0 |
| | No | 0 | 0.0 |
| | Indifferent | 1 | 4.5 |
| thoroughly cleaned. | Yes | 20 | 91.0 |
| | Excellent | 1 | 4.5 |
| The scalp seems to become fresh due to reduced amount of excess sebum. | Not at all | 0 | 0.0 |
| | No | 0 | 0.0 |
| | Indifferent | 2 | 9.1 |
| | Yes | 16 | 72.7 |
| | Excellent | 4 | 18.2 |
| Total | | 22 | 100.0 |

Additionally, a survey was conducted by asking whether there are any adverse reaction to the scalp and hair when using the composition of Examples. The results were shown in Table 15 below (0: none, 1: mild, 2: moderate, 3: severe).

TABLE 15

| Adverse reaction | Immediately after a single use | Adverse reaction | Immediately after a single use |
|---|---|---|---|
| Erythema (redness) | 0 | Stinging (pain) | 0 |
| Edema (swelling) | 0 | Burning sensation | 0 |
| Scale (keratin) | 0 | Stiffness | 0 |
| Itchiness | 0 | Tingling | 0 |

As shown in Tables 11 and 12, after a single application of the hair shampoo according to Example 1-1 of an embodiment of the present disclosure, the amount of sebum was improved by 80% or higher compared to before, showing that the scalp oil improvement effect was excellent. These results were consistent with the results of the questionnaire on the test subjects shown in Tables 13 to 15 in that more than 90% of the test subjects answered that there were cleaning and sebum removal effects without side effects.

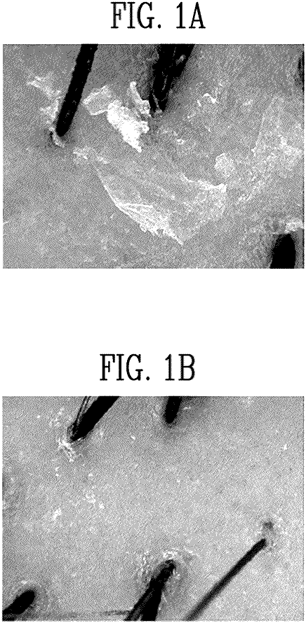

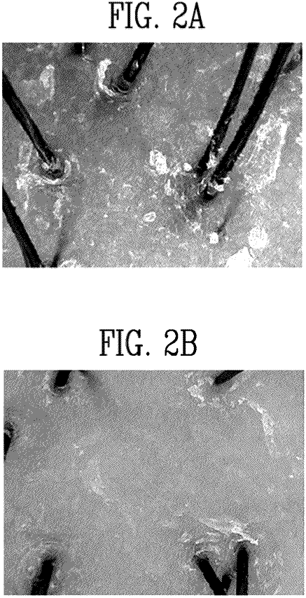

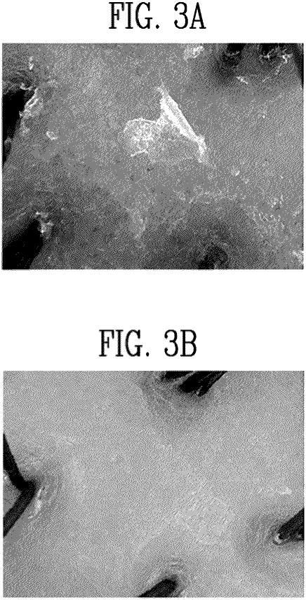

FIGS. 1 to 3 are photographs showing enlarged observation results (A) before application and (B) after a single application of the hair shampoo according to Example 1-1 of an embodiment of the present disclosure for three test subjects, showing that the cleaning and sebum removal effects were excellent.

### Experimental Example 6. Checking Whether the Scalp Flora Is Improved

The effect of improving the scalp flora was identified when the hair shampoo composition according to Example 1, the hair treatment composition according to Example 2, and the hair tonic composition according to Example 3 were used. As the test subjects, 24 adult women with an average age of 45 were chosen, whose scalp was selected as the test site. The test subjects were not allowed to use other products such as shampoos, treatments, and hair essences and get hair dyed or permed during the experiment.

The experiment was performed after allowing the test subjects to take a rest for 30 minutes in a constant temperature and humidity room at 20 to 25° C. and 40 to 60% humidity, and all experiments were performed in the constant temperature and humidity room. After the hair and scalp of the test subjects were sufficiently wet with lukewarm water, the hair shampoo composition according to Examples 1-1 and 1-2 and the hair treatment composition according to Example 2 were evenly applied to the hair and scalp in the same amount once a day, respectively. After massage, the test subjects were directed to rinse thoroughly with running water. In addition, the test subjects were directed to evenly apply the same amount of the hair tonic composition according to Example 3 to the scalp once a day. After massage, the applied hair tonic composition was left to be dried without rinsing.

After the experiment period, without applying shampoo and water to the hair and scalp for a day, the test subjects were allowed to take a rest for 30 minutes in the constant temperature and humidity room. Microorganisms on the scalp were collected by swabbing the scalp with the cotton swab up and down 40 times while the middle line of the head is fixed so as to compare and analyze with the microorganisms collected before the experiment.

Hereinafter, the analysis was conducted using SPSS for statistical processing in this experiment. As a result of the analysis, it was determined that there was an improvement effect when the significance probability is greater than 0.05 (p<0.05) in the 95% confidence interval. As for the statistical analysis, the analysis was conducted using paired samples t-test when a parametric method was applied and Wilcoxon signed rank test when a nonparametric method was applied. Table 16 shows results of analysis of variance (ANOVA) at the genus level before and after use of the composition according to Examples.

TABLE 16

| Genus | p value | Before use | 1 week after use | 2 weeks after use |
|---|---|---|---|---|
| Aspergillus | p < 0.01 | 2.21 | 2.01 | 0.78 |
| Fusarium | p < 0.01 | 1.58 | 1.49 | 0.28 |
| Penicillium | p < 0.01 | 1.12 | 0.99 | 0.21 |
| Cladosporium | p < 0.01 | 1.03 | 0.98 | 0.19 |
| Mucor | p < 0.01 | 0.66 | 0.71 | 0.2 |
| Alternaria | p < 0.01 | 0.6 | 0.56 | 0.13 |
| Kluyveromyces | p < 0.01 | 0.66 | 0.54 | 0.06 |
| Clitocybe | p < 0.01 | 0.6 | 0.57 | 0.032 |
| Sarocladium | p < 0.01 | 0.53 | 0.55 | 0 |

After 2 weeks of using the composition according to Example, it was determined that the flora of 9 genera existing on the scalp was statistically significantly changed. As shown in Table 16, from 1 week after using the compositions according to Examples 1 to 3 of an embodiment of the present disclosure, the genus Saccharomyces, known to contribute to scalp moisturization and elasticity improvement, increased statistically significantly. In the genera of Aspergillus, Fusarium and Penicillium, known to cause mycoses or dermatitis, it was found that the flora was statistically significantly reduced after 2 weeks of use, meaning that the flora was statistically significantly improved in the scalp.

### Experimental Example 7. Checking Whether Skin Flora is Improved

The skin flora improvement effect was identified when the body wash composition according to Example 4 and the body lotion composition according to Example 5 were used. As the test subjects, 22 adult women aged 20 to 60 years were chosen, whose skin of the forearm was selected as the test site. The test subjects were not allowed to use other body products during the experimental period.

The experiment was performed after the test subjects were allowed to take a rest for 30 minutes in a constant temperature and humidity room at 20 to 25° C. and 40 to 60% humidity without washing the test site for more than 8 hours, and all experiments were carried out in the constant temperature and humidity room. The skin of the forearm of the test subjects was wet with lukewarm water, washed with the body wash composition according to Example 4, and evenly applied with the body lotion composition according to Example 5.

After the experiment period, the microorganisms were collected by swabbing the skin of the forearm with the cotton swab up and down 40 times, and compared and analyzed with the microorganisms collected before the experiment. Statistical processing was performed in the same manner as in Experimental Example 6 described above. Table 17 shows the results of analysis of variance (ANOVA) at the genus level before and after the use of the composition according to the Example.

TABLE 17

| Genus | p value | Before use | 1 week after use | 2 weeks after use |
|---|---|---|---|---|
| Ehrlichia | p < 0.001 | 7.43 | 7.54 | 2.62 |
| Sphingomonas | p < 0.001 | 3.19 | 3.22 | 2.29 |
| Pseudomonas | p < 0.001 | 1.56 | 1.5 | 3.31 |
| Acetobacter | p < 0.001 | 2.65 | 2.7 | 0.95 |
| Cutibacterium | p < 0.001 | 1.22 | 1.17 | 3.07 |
| Streptococcus | p < 0.001 | 1.13 | 0.96 | 1.93 |
| Akkermansia | p < 0.001 | 1.55 | 1.56 | 0.85 |
| Bacteroides | p < 0.001 | 1.37 | 1.41 | 0.69 |
| Staphylococcus | p < 0.001 | 0.72 | 0.72 | 1.81 |
| Enterococcus | p < 0.001 | 0.93 | 0.92 | 0.80 |

After 2 weeks of using the composition according to the Example, it was found that the flora of 10 genera on the skin was statistically significantly changed. As shown in Table 17, the Staphylococcus and Cutibacterium genera increased statistically significantly after 2 weeks of use of the compositions according to Examples 4 and 5 of an embodiment of the present disclosure, and Ehrlichia and Sphingomonas genera, known to cause infections, were statistically significantly reduced after 2 weeks of use. Thus, the flora in the skin was statistically significantly improved. In addition, as a result of measuring the diversity of the flora (β-diversity), a statistically significant increase was observed, determining that the flora in the skin was statistically significantly improved.

The present disclosure is not limited to the examples described above, and a combination of the examples or a combination of at least one of the examples and a known technology may be included as another example.

Although the present disclosure has been described in detail through specific examples, this is for the purpose of describing the present disclosure in detail, and the present disclosure is not limited thereto. It will be apparent that modifications or improvements are possible by those of ordinary skill in the art within the technical spirit of the present disclosure.

All simple modifications or changes of the present disclosure fall within the scope of the present disclosure, and the specific protection scope of the present disclosure will become apparent by the appended claims.

1-17. (canceled)

18. A cosmetic composition for skin improvement, comprising a polysaccharide, a yeast extract, and a strain fermentation product with probiotic characteristics as active ingredients.

19. The cosmetic composition of claim 18, wherein the polysaccharide is one or more selected from the group consisting of inulin, beta-glucan, and maltodextrin.

20. The cosmetic composition of claim 18, wherein the yeast extract is one or more selected from the group consisting of a beer-derived yeast extract and a truffle-derived yeast extract.

21. The cosmetic composition of claim 18, wherein a strain with the probiotic characteristics is one or more selected from the group consisting of Lactobacillus sp. strain and Bifidobacterium sp. strain.

22. The cosmetic composition of claim 21, wherein the fermentation product is one or more selected from the group consisting of a ferment lysate and a ferment filtrate.

23. The cosmetic composition of claim 18, comprising inulin, beta-glucan, maltodextrin, a beer-derived yeast extract, a truffle-derived yeast extract, a Lactobacillus ferment lysate, and a Bifidobacterium ferment lysate as active ingredients.

24. The cosmetic composition of claim 23, comprising inulin, beta-glucan, maltodextrin, the beer-derived yeast extract, the truffle-derived yeast extract, the Lactobacillus ferment lysate, and the Bifidobacterium ferment lysate in the same weight ratio, respectively.

25. The cosmetic composition of claim 23, comprising one or more active ingredients selected from the group consisting of inulin, beta-glucan, maltodextrin, the beer-derived yeast extract, the truffle-derived yeast extract, the Lactobacillus ferment lysate, and the Bifidobacterium ferment lysate in an amount of 0.00001 to 10 wt% with respect to 100 wt% of the cosmetic composition.

26. The cosmetic composition of claim 18, wherein the cosmetic composition is for improving microbial flora inhabiting the skin.

27. The cosmetic composition of claim 26, wherein the cosmetic composition is for promoting growth of beneficial bacteria inhabiting the skin or suppressing growth of harmful bacteria.

28. The cosmetic composition of claim 27, wherein the beneficial bacteria is Staphylococcus epidermidis (S. epidermidis), and
the harmful bacteria is Staphylococcus aureus (S. aureus).

29. The cosmetic composition of claim 18, wherein the cosmetic composition is for any one of skin soothing, skin wrinkle amelioration, and skin elasticity improvement.

30. The cosmetic composition of claim 18, wherein the skin is scalp.

31. The cosmetic composition of claim 30, wherein the cosmetic composition is for any one of scalp soothing, scalp oil improvement, hair loss prevention, and hair growth promotion.

32. The cosmetic composition of claim 30, wherein the cosmetic composition is for improving microbial flora inhabiting the scalp.

33. A method for improving skin, comprising applying, to the skin, a cosmetic composition for skin improvement including a polysaccharide, a yeast extract, and a strain fermentation product with probiotic characteristics as active ingredients.

34. The method of claim 33, wherein the polysaccharide is one or more selected from the group consisting of inulin, beta-glucan, and maltodextrin.

35. The method of claim 33, wherein the yeast extract is one or more selected from the group consisting of a beer-derived yeast extract and a truffle-derived yeast extract.

36. The method of claim 33, wherein a strain with the probiotic characteristics is one or more selected from the group consisting of Lactobacillus sp. strain and Bifidobacterium sp. strain.

37. The method of claim 33, wherein the method is any one of a method for improving microbial flora inhabiting the skin, a skin soothing method, a skin wrinkle amelioration method, a skin elasticity improvement method, a method for improving microbial flora inhabiting scalp, a scalp soothing method, a scalp oil improvement method, a hair loss prevention method, and a hair growth promotion method.
